(19) United States
(12) Patent Application Publication
(10) Pub. No.: US 2007/0172474 A1
(43) Pub. Date: Jul. 26, 2007

(54) Dairy components effective for fat loss

(75) Inventor: Michael B. Zemel, Knoxville, TN (US)

Correspondence Address: VENABLE LLP, P.O. BOX 34385, WASHINGTON, DC, 20043-9998, US

(73) Assignee: University of Tennessee Research Foundiation, Knoxville, TN (US)

(21) Appl. No.: 11/547,070

(22) PCT Filed: Mar. 29, 2005
(86) PCT No.: PCT/US05/10098

§ 371 Date: Oct. 2, 2006

Related U.S. Application Data
(60) Provisional application No. 60/558,706, filed on Apr. 2, 2004.

(51) Int. Cl. A61K 39/395; A61K 35/20

(52) U.S. Cl. 424/130.1; 424/535

(57) ABSTRACT

The present invention relates to methods comprising administering to an individual regulating body weight calcium-containing whey products in an amount effective to maintain muscle mass, induce weight and/or fat loss, prevent weight and/or fat gain, and/or increase the metabolic consumption of adipose tissue in the individuals.

### FIELD OF THE INVENTION

The present invention relates to use of whey products in weight management. In particular, the invention encompasses a method comprising administering to an individual regulating body weight an amount of a calcium-containing whey product effective to maintain muscle mass while inducing weight loss, preventing weight gain, and/or increasing the metabolic consumption of adipose tissue.

### BACKGROUND OF THE INVENTION

Dietary calcium has been shown to induce weight loss, prevent weight gain and/or increase the metabolic consumption of adipose tissue. See e.g. U.S. Pat. No. 6,384,087, Zemel, et. al., which is incorporated herein by reference. It was not previously recognized that such effects can be achieved by administering whey products.

However, individuals often gain 1-3 pounds of weight a year and when dieting, although fat loss may be achieved, it is often accompanied with loss of lean or muscle mass. There is a need for providing diets that selectively target specific fat zones while maintaining lean or muscle mass.

The present invention is directed to products containing ingredients from whey that augment weight loss while protecting muscle mass.

### SUMMARY OF THE INVENTION

The present invention relates to, e.g., methods comprising administering to an individual regulating body weight a calcium containing whey product in an amount effective to induce weight and/or fat loss, prevent weight and/or fat gain, and/or increase the metabolic consumption of adipose tissue in the individual, while maintaining muscle mass.

### BRIEF DESCRIPTION OF THE DRAWINGS

FIG. 1 is a schematic illustration of the effects and mechanism of whey products. Increased intracellular calcium ([Ca²⁺]ᵢ) in human adipocytes stimulates lipogenesis and inhibits lipolysis, thereby increasing triglyceride accumulation. In contrast, increasing dietary calcium and dairy suppresses calcitrophic hormone 1α,25-(OH)₂-D₃, thereby reducing [Ca²⁺]ᵢ and, consequently, triglyceride accumulation.

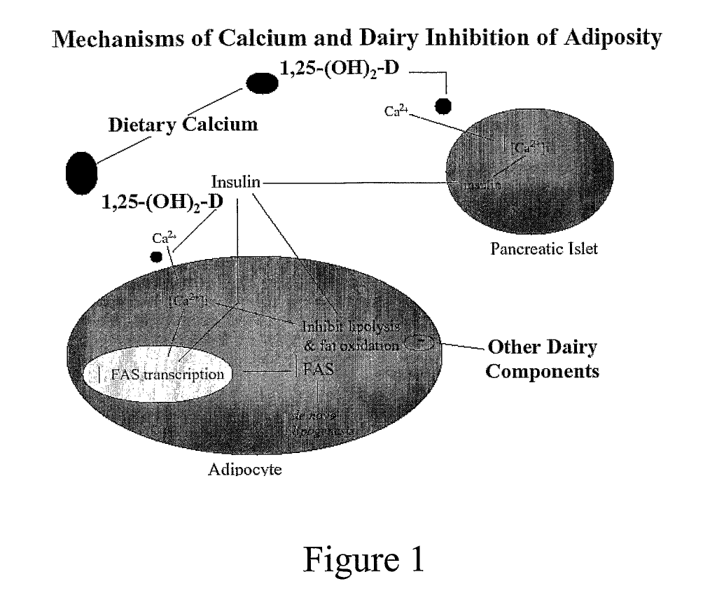

FIG. 2 outlines the specific feeding pattern of calcium and selective dairy components in eight groups of transgenic mice expressing agouti specifically in adipocytes under the control of the aP2 promoter.

FIG. 3 shows the effects of consuming calcium and selective dairy components for six weeks on weight change in transgenic mice expressing agouti specifically in adipocytes under the control of the aP2 promoter.

FIG. 4 illustrates the effects of consuming calcium and selective dairy components for six weeks on Fat Pad Mass in transgenic mice expressing agouti specifically in adipocytes under the control of the aP2 promoter.

FIG. 5 depicts the effects of consuming calcium and selective dairy components for six weeks on Abdominal Fat in transgenic mice expressing agouti specifically in adipocytes under the control of the aP2 promoter.

FIG. 6 shows the effects of consuming calcium and selective dairy components for six weeks on Perirenal Fat in transgenic mice expressing agouti specifically in adipocytes under the control of the aP2 promoter.

FIG. 7 illustrates the effects of consuming calcium and selective dairy components for six weeks on Subscapular Fat in transgenic mice expressing agouti specifically in adipocytes under the control of the aP2 promoter.

FIG. 8 depicts the effects of consuming calcium and selective dairy components for six weeks on Gastrocnemius Mass in transgenic mice expressing agouti specifically in adipocytes under the control of the aP2 promoter.

### DESCRIPTION OF THE INVENTION

The calcium-containing whey products of the present invention may be whey or a composition containing whey, whey-derived protein product, whey-derived mineral isolate blends, or combinations thereof, any of which may optionally include intrinsic or additional enzyme-inhibiting peptides, such as angiotensin converting enzyme (ACE) inhibiting peptides.

Whey is generally known as the watery part of milk that is separated from the coagulable part or curd especially in the process of making cheese (Merriam-Webster Online Dictionary<www.m-w.com>). Whey is readily available as a by-product from the dairy industry, which typically is prepared by removing fat and casein from milk, and contains lactose, some salts, and a variety of proteins, including bovine serum albumin “BSA” (molecular weight 66,000 Daltons), alpha-lactoglobulin (molecular weight 14,000 Daltons), beta-lactoglobulin (molecular weight 16,000 Daltons), kappa-casein fragment 106-109, lactoferrin, lactoperoxidase, and immunoglobulins. Salts contained in whey or milk may include calcium, copper, magnesium, phosphorus, potassium, selenium and/or zinc. Whey protein preparations are commercially available, for example, as whey protein concentrates (WPC), which comprises protein and carbohydrate (lactose), e.g., AVONLAC 134 (Glanbia) or whey protein isolates (WPI), e.g., PROVON 190 or 290 (Glanbia). Other whey providers are Davisco (Le Sueur Minn.); Bio-Isolates PLC (Deeside, UK); NZMP North America (Santa Rosa Calif.); Formost Farms (Baraboo Wis.); Arla Foods (Union N.J.); Avenmore Waterford (Monroe Wis.); Borculo Whey Products; and Borculo, Netherlands.

A whey product according to the invention may be any such fraction derived from any dairy product such as milk, cream, or cheese whey. The whey product maybe any product produced from whey after fractionation or isolation from milk, including whey, WPC, WPI, whey mineral concentrate, such as TRUCAL calcium isolate (Glanbia), containing 24% calcium and 13% phosphorus. The whey product may be obtained by any method known in the art. For example, whey fractions may be obtained by one or more of extraction, ultrafiltration, electrodialysis, evaporation, and reverse osmosis of milk or cheese whey.

The whey product may be in a composition that includes enzyme-inhibiting peptides, which may be derived from such peptides as casein, whey proteins, soy proteins, or any other suitable source via processing means known to those of skill in the art. One example of such peptide is one that inhibits angiotensin converting enzyme (ACE). For example, TRUCAL FP (Glanbia) contains dairy mineral and ACE inhibiting peptide.

The whey product may be administered to an individual in any form known in the art to carry out the purpose of the invention.

In an exemplary embodiment, the whey product is a composition containing whey-derived protein isolate with about 25% to about 99% protein, or at least about 75%, 80%, 85%, 90%, 95%, or greater of protein. The composition may additionally contain less than about 10%, 5%, or 1% of carbohydrates, lactose, fat or other minerals. In another aspect, the protein iso late may contain stabilizers or emulsifiers such as lecithin, (ethoxylated mono diglycerides, and propylene glycol).

In an exemplary embodiment, the whey product is a composition containing whey-derived mineral fraction comprising from about 1-99% whey derived mineral comprising from about 1-30%, 10-90%, 20-80%, 30-70%, 15-30%, 20-30%, 22-28%, or 23-25% calcium.

In another exemplary embodiment, the whey product is a composition containing whey-derived mineral fraction and whey-derived protein isolate, e.g., in the above-stated amounts, for example at least about 78% protein, at least about 15% total minerals and at least about 3.8% calcium.

In an exemplary aspect of the embodiment, the whey product fortifies calcium-containing products. Examples of calcium-containing products include dietary calcium, calcium carbonate preparations, dairy or a product derived from dairy, such as milk, yogurt or cheese. The calcium-containing product may be, e.g., yogurt or a product derived from yogurt, cheese or a product derived from cheese, or milk or a product derived from milk, such as skim milk, 1% milk, 2% milk, whole milk, half and half or whipping cream. In another exemplary embodiment, the whey product is in the form of a powder or is incorporated into a nutritional or dietary composition or supplement or calcium fortified vitamin supplements, or is incorporated into a food product or foodstuff or other foods high in calcium or any food product that is consumed by the individual. The foodstuff maybe cereal, salmon, beans, tofu, spinach, turnip greens, kale, broccoli, waffles, pancakes, pizza, cottage cheese, ice cream or frozen yogurt. In another exemplary embodiment, the calcium-containing product comprises infant formula, nutriceuticals, or meal replacement beverages or drinks or powders for preparing any of the above.

In another embodiment, the product may be in the form of a pill, tablet, capsule, or combination with other minerals and/or vitamins. The product may be for human or non-human/animal consumption, such as pet food or farm/agricultural animal feed.

The present invention also provides methods of increasing the amount of whey product ingested or consumed by the individual and, optionally, in combination with other dietary efforts, e.g., restricting the caloric intake or exercising, and/or administering the whey product for a prolonged period of time to obtain the desired results of inducing weight and/or fat loss, preventing weight and/or fat gain, and/or increasing the metabolic consumption of adipose tissue. For example, the product may be administered for a continuous interval of at least about one week, two weeks, three weeks, one month, two months, three months, six months, or one year, wherein the product is administered on an average daily basis in amounts effective to induce the desired results.

The amount effective to induce the desired results may be based on the amount of calcium contained in said whey product, e.g., at least about 1000 mg calcium per day. A whey product or a serving thereof may contain on average at least about 100 mg, 200 mg, 255 mg, 300 mg, 400 mg or 500 mg. Whey product consumption may provide calcium consumption of at least about 500 mg, 600 mg, 773 mg, 800 mg, 900, mg, 1000 mg, 1100 mg, 1200 mg, 1300 mg, 1346 mg, 1400 mg, 1500 mg, 1600 mg, or higher amounts, on average, for at least two weeks, one, two, three months, or longer. In an exemplary embodiment, the whey product is administered over a continuous interval for at least one month, providing at least about 1000 mg of calcium on an average daily basis. These dosages may be referred as being “high calcium.”

Another aspect of the invention provides for methods of determining dietary calcium consumption of the individual, wherein the individual is a human and (1) if the dietary calcium consumption is below 1000 mg/day, increasing the whey product consumption, and (2) if the dietary calcium consumption is at least about 1000 mg/day, maintaining that level through whey product consumption. In another embodiment the amount of whey product consumed by the individual before administering the effective amount of whey product is less than about 400 mg/day, 600 mg/day or 773 mg/day of calcium. In another aspect, the average daily whey product administered may provide at least about, 800 mg/day, 1000 mg/day, 1100 mg/day, 1200 mg/day, 1346 mg/day, or 1400 mg/day of calcium.

In yet another exemplary embodiment, the effective amount of calcium-containing whey product may be in the form of a dairy product fortified with the whey product which is administered in at least about 3 to 4 servings per day, e.g., 2.5 or 3.5 servings. An example of serving size may comprise whey fortified product having the calcium content of at least about 8 ounces of milk, 8 ounces of yogurt or 1.5 ounces of cheese. In an exemplary embodiment, a serving may contain at least about 200, 255 or 300 mg, two servings may contain about 700, 773 or 800 mg, and three servings may contain 1000, 1100, 1300, 1346 or 1400 mg of calcium. In one aspect, the serving portion may contain on average at least about 300 mg of whey-derived dietary calcium. In another aspect of the invention, a serving portion may contain on average at least about 200 mg of dietary calcium. In an exemplary embodiment, the effective amount of whey-derived dietary calcium is at least about 1000 mg per day. In another embodiment, the whey product may be administered on an average daily basis equaling to at least about 50 to 75 servings per month or 100 to 120 servings per month. In an embodiment, the amount of calcium is administered on average at about 1346 mg a day, or 3 to 3.5 servings ofwhey product to achieve the desired effects of inducing weight and/or fat loss, preventing weight and/or fat gain, and/or increasing the metabolic consumption of adipose tissue.

The whey product of the present invention provides high calcium bioavailability and is more effective than mineral calcium for regulating body weight and/or body fat by inducing weight and/or fat loss, preventing weight and/or fat gain, and/or increasing the metabolic consumption of adipose tissue in the individual.

The methods and compositions of the invention are effective at regulating or controlling body weight and adiposity. They are also effective in preventing, reducing the severity of, reducing the risk of, or treating a body weight disorder or other disorder that is associated with being overweight such as obesity, high blood pressure, stroke, premenstrual syndrome, depression, hypertensive disorders of pregnancy, Type-2 diabetes, asthma, inflammatory bowel disease, attention deficit disorder, migraine headaches, kidney disease, kidney stones, hypercholesterolemia, congestive heart failure, immune deficiency, and others known to practitioners.

The inventors previously discovered that calcium-containing products, such as milk, are effective for inducing weight and/or fat loss. As set forth and detailed in Example 1, surprisingly, calcium-containing whey products are more effective than calcium in the same amounts from mineral sources, such as calcium carbonate, at inducing weight loss, preventing weight and/or increasing the metabolic consumption of adipose tissue in an animal. Diets with whey, whey protein, whey derived mineral isolate or blends, or whey derived mineral isolate with enzyme-inhibiting peptides, can be more effective than administering calcium alone or calcium with other non-whey containing products in inducing weight and/or fat loss.

The present invention may also include maintaining the individual on a caloric restricted or energy restricted diets. Often such restricted diets results in both fat and muscle loss. The present invention provides for a method of attaining weight regulation by selectively targeting specific regions of the body and/or types of fat or muscle cells or tissue.

In an exemplary embodiment, the present invention is directed to methods of providing weight related diets, e.g., that selectively target specific fat cell or tissue zones while maintaining lean or muscle mass, by administering whey products to an individual and thereby augmenting weight loss, while protecting muscle. That is, individuals lose more fat and less lean. The invention may also help the body recover rapidly from the metabolic stress of exercise and help individuals avoid gaining extra pounds upon cessation of a weight loss diet. These whey-based diets promote a repartitioning of dietary energy from adipose tissue to skeletal muscle, resulting in preservation and/or improvement in lean body mass while adipose tissue is lost.

For example, one embodiment provides a method comprising administering to an individual regulating body weight a calcium containing whey product in an amount effective to maintain muscle mass while inducing weight and/or fat loss, preventing weight and/or fat gain, and/or increasing the metabolic consumption of adipose tissue in the individual.

The retention of lean or muscle mass maybe maintained selectively, in various regions of the body, e.g., skeletal muscle in limbs (legs or arms) or the trunk. As shown below in Example 1, in a mouse model predictive of human effects, gastrocnemius (leg) muscle is maintained. Likewise, inducement of fat loss may also be selectively targeted in various regions or types, e.g., loss of fat pad mass, abdominal fat, perirenal fat or subscapular fat and/or combinations. The present invention may assist in inducing fat loss while maintaining muscle mass, that is increasing lean or muscle mass by at least about 1, 2, 5 or 10%, or resulting in loss of muscle mass of no more than about 1, 2, 5, or 10%.

The efficacy of diets containing various constituents for limiting weight and fat was measured to be approximately as follows, from least to most effective constituents: low calcium, high mineral calcium, whey mineral isolate, whey protein, and milk. ACE inhibitors improved the efficacy of whey mineral isolate and whey protein diets.

Example 1 suggests whey mineral calcium isolate is not as effective as milk mineral calcium, but this result is apparently limited to that particular source of whey mineral (Glanbia TRUCAL and TRUCAL FP). Other sources of whey calcium or milk-derived calcium are at least as effective at regulating adiposity as other supplementary calcium sources (e.g. calcium carbonate).

The data show that whey surprisingly retains most or all of the bioactivity found in milk and that the whey-derived ACE inhibitor contributes to this effect. The ACE inhibitory action causes a consistent improvement in adiposity, weight loss, etc., but intact dairy or intact whey has a much larger effect.

In another exemplary embodiment, the invention is directed to compositions and methods for maintaining a predetermined body weight range and treating an overweight condition or obesity by enhancing weight loss and/or limiting weight gain and promoting good health. Overweight and obesity has been associated to some degree with inadequate intake of dairy products, and more particularly the minerals present in dairy products. It has been discovered that an overweight condition and obesity can be effectively treated by administering nutritional supplement compositions, either directly or via food products fortified with the compositions, in accordance with the practice of the present invention. The nutritional supplement compositions contain therapeutically effective amounts of whey mineral, whey protein and/or whey-derived enzyme-inhibiting peptides and are administered prior to or during a meal. The nutritional supplement compositions also can be administered to an individual seeking to maintain a desired body weight.

Treatment can be enhanced by use of additional ingredients in the composition to address other mechanisms for weight control. Additionally, enzyme-inhibiting peptides maybe included to assist with regulation of adiposity by controlling fat metabolism.

The terms “treat,” “treating,” “treatment,” and similar terms as used herein refer to the administration of the nutritional supplement compositions to individuals, particularly humans, who are overweight or obese, for alleviating, suppressing, inhibiting, or otherwise reducing the extent to which the individual is overweight or obese or any symptom associated therewith. The terms “treat,” “treating,” “treatment,” and similar terms also are used herein to refer to the prophylactic administration of the nutritional supplement compositions to individuals who maybe at risk of, or otherwise wish to avoid, becoming overweight or obese.

One component of the nutritional supplement composition is whey minerals. The term “whey mineral,” as used herein, refers to a mineral complex obtained from whey. The mineral complex contains a balanced form of calcium, copper, magnesium, phosphorus, potassium, selenium and zinc. Whey fractions that are high in calcium have been demonstrated to exhibit higher calcium bioavailability than are exhibited by calcium carbonate and calcium lactate. Ranhotra et al., “Bioavailability of Calcium in a High Calcium Whey Fraction,” Nutrition Research, Vol. 17 Nos. 11-12, pp. 1663-1670(1997). The whey mineral fraction also typically contains quantities of lactose and bioactive proteins. Whey mineral is also commonly referred to as “milk calcium” or “milk mineral.”

Whey mineral provides various benefits as compared to supplements having other forms of calcium. Calcium supplements and calcium-fortified foods contain calcium in such forms as calcium carbonate, calcium lactate, calcium citrate, calcium chloride, and calcium hydroxide. These forms of calcium, however, can yield undesirable flavors and/or can strip desirable aroma and flavor compounds from food products. Use of whey minerals can avoid these problems. The mineral complex provides a balanced form of minerals, including calcium that is a preferred form of calcium and other minerals from a nutritional standpoint.

Suitable methods of obtaining mineral complex by extraction from whey or milk are known to persons skilled in the art. One suitable extraction method is described in U.S. Pat. No. 5,639,501, the disclosure of which is incorporated by reference in its entirety. Additionally, commercially available milk mineral products include TRUCAL products (Glanbia Nutritionals, Inc. of Monroe, Wis.). A typical composition of whey mineral is illustrated in Table 1 below.

TABLE 1
Typical Composition for Whey Mineral Powder

| | |
|---|---|
| Component | Relative Amount (% by weight) |

| | |
|---|---|
| Total Minerals | 50-90% |
| Inorganic Mineral (Ash) | 45-85% |
| Organic Mineral (Citrate) | 1-10% |
| Calcium | 15-35% |
| Magnesium | 0-10% |
| Phosphorous | 7-15% |
| Potassium | 0-5% |
| Zinc | 0-1% |
| Lactose | 0-15% |
| Protein | 1-15% |
| Free Moisture | 2-5% |
| Fat | 0-5% |

The balance of minerals and bioactive proteins in the mineral fraction renders food products fortified with the compositions of the present invention effective for healthy weight maintenance and in the treatment of the conditions of overweight and obesity. While not wanting to be bound by any theory, the following provides a discussion of the various mechanisms by which body weight conditions can be treated using the compositions of the present invention.

The whey minerals of the present compositions provide high calcium bioavailability effective for managing body weight and treating conditions including overweight and obesity. A common metabolic defect in cellular calcium ion handling is thought to contribute to the occurrence of weight gain and obesity. Low calcium intake increases intracellular calcium concentration in the adipocyte (fat cell) thereby switching its metabolism from lipolysis (fat breakdown) to lipogenesis (fat synthesis) and fat accumulation. By increasing the amount of calcium intake, intracellular calcium concentration is reduced, which leads to increased lipolysis and decreased lipogenesis. Thus, it is desired to provide a daily intake of an amount of calcium effective for weight maintenance and/or loss through reduction of fat tissue mass.

In another aspect of the present invention, the nutritional supplement composition includes a protein source such as whey proteins. Whey proteins occur in milk as soluble, globular proteins and are different than the suspended casein particles. Generally, they are an important source of protein needed for overall good health and nutrition. The primary proteins and peptide constituents derived from whey proteins include alpha-lactalbumin and beta-lactoglobulin, kappa-casein fragment 106-109, lactoferrin, bovine serum albumin, lactoperoxidase, and immunoglobulins.

In another aspect of the present invention, the composition includes enzyme-inhibiting peptides. Sources of such peptides include casein, whey proteins, soy proteins, or any other suitable commercially available food protein which can be processed according to any methods known to those of skill in the art to provide the peptides. One example of such peptides are those that inhibit angiotensin converting enzyme (ACE). Angiotensin II is a hormone that is synthesized and secreted by adipose cells. Literature has shown that angiotensin II may be involved in control of adiposity through regulation of lipid synthesis and storage of adipocytes. Some dairypeptides are associated with the inhibition of angiotensin converting enzyme (ACE). Thus, by administering a therapeutically effective amount of ACE-inhibiting peptides, weight loss can be enhanced. One or more enzyme-inhibiting peptides can be included in the composition.

To obtain the compositions of the present invention, the components selected for the compositions can be processed as desired prior to preparation of the nutritional supplement compositions. Whey mineral extract typically is purified, spray dried, and ground into a powder having an appropriate particle size to permit mixing with a liquid or solid food product if desired. The mineral extract has calcium and other minerals as shown in Table 1. Similarly, the protein component typically is purified, dried, and ground into a powder. ACE-inhibiting peptides also can be provided in the nutritional supplement composition. The desired components, which are selected from milk mineral extract, protein component and ACE-inhibiting peptides and combinations thereof, are blended to provide the nutritional supplement compositions of the present invention. The composition optionally can include other ingredients, such as minerals, vitamins, flavorings and colorants, in accordance with techniques well known to persons skilled in the art.

Suitable particle sizes for the composition will depend on such factors as the physical properties (e.g., liquid or solid, specific gravity, pH, viscosity, etc.) of the food product into which the powder is mixed.

The nutritional supplement composition in powder form can be used as an additive for a wide variety of types of food products, and food product compositions containing whey products according to the invention include acidic juice beverages (e.g., orangejuice, applejuice, grapejuice, grapefruit juice, cranberry juice, or blended juices), acidic beverages (e.g., sport beverages, GATORADE), neutral pH beverages (e.g., milk UHT dairy, RTD nutritional, soy milk, or shakes and other blended beverages such as milkshakes, smoothies, frappes), nutritional supplement foodstuffs (e.g., high-energy protein bars), confectionery products (e.g., high calcium chews, chewing gum, chocolate, or cookies), dairy products (e.g., yogurt, ice cream, milk, cheese, processed cheese, or butter), and farinaceous products (e.g., bread, muffins, biscuits, cereal or rolls). The relative amount by weight of the nutritional supplement compositions combined with a food product depends on such factors as the density and the serving size of the food product. Typically, the amount of nutritional supplement compositions ranges from 0.1 to about 10 percent by weight, based on the total weight of the food product.

The formulation of the composition and, if administered via a food product, the amount of the composition blended into the food product, are selected to provide desired amounts of the particular components so as to be effective for controlling weight gain and/or weight loss. By way of example, a typical nutritional supplement composition may be administered to provide between at least about 0.5 and about 6 grams or more of calcium, and at least about 0.1, 0.5, 1, 2, 3, 5, 10 or 20 grams or more of ACE-inhibiting peptides per serving of the composition. The amount of composition administered can be adjusted as desired to account for differences in physical characteristics and nutritional requirements of the individuals to whom the composition is administered.

In accordance with the methods of the present invention, body weight conditions, including overweight and obesity, are effectively managed and treated. That is, an individual of healthy condition and having a generally ideal weight can manage his or her weight and maintain a desired weight range. An individual who has a weight in excess of a desired range, and may be considered overweight or obese, can be effectively treated by limiting weight gain and/or promoting weight loss while maintaining muscle mass. A therapeutically effective amount of the nutritional supplement composition is administered to an individual to provide these benefits.

### EXAMPLES

### Example 1

Objective

The objective of this example is to determine the fraction(s) of dairy responsible for dairy-induced augmentation of the anti-obesity muscle mass retaining effect of calcium in diet-induced obese transgenic mice. Specifically, it is to determine the effectiveness ofwhey-based high calcium diets versus casein-, soy- and milk-based high calcium diets (compared to a low calcium control) in accelerating weight and fat loss secondary to caloric restriction in agouti-transgenic mice fed high fat/high sucrose/low fat diets, and to whether the augmented anti-obesity effect of whey is attributable to whey protein(s) or the mixture of minerals, in addition to calcium, found in whey.

Animal and Diets

To evaluate the role of dietary calcium in regulating adiposity in vivo, transgenic mice expressing agouti specifically in adipocytes under the control of the aP2 promoter were studied. These animals exhibit a normal pattern of leptin expression and activity similar to that found in humans and exhibit a human pattern (adipocyte-specific) of agouti expression. These mice are useful models for diet-included obesity in that they are not obese on a standard AIN-93G diet, but become obese in response to hyperinsulinemia induced by either insulin administration or high sucrose diets.

Methods

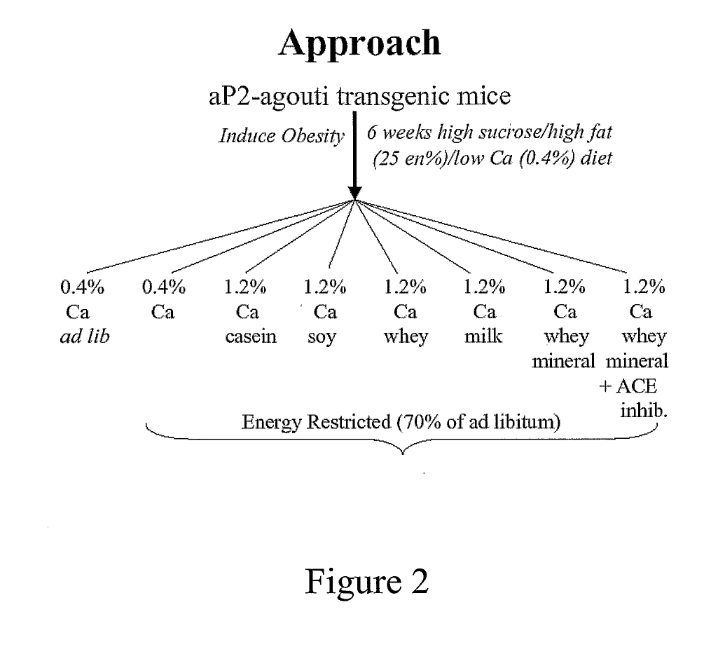

As illustrated in FIG. 2, mice were fed a low calcium (0.4%), high sucrose, high fat diet for 6 weeks to induce obesity. All diets had 25% fat (lard+soy oil), with sucrose as sole carbohydrate and the proteins indicated as the sole protein source. Group 1 continued for 6 weeks on the above diet ad libitum.

Groups 2-8 continued for six weeks on a restricted (70% of the ad libitum energy intake). Group 2 was maintained at a low calcium diet (casein/0.4% calcium) comprising protein from milk casein and 0.4% calcium from CaCO₃.

Groups 3-6 continued on high calcium, energy restricted diets. Group 3 was maintained on non-fat dry milk/1.2% calcium comprising dried milk having 1.2% calcium, most of which comes from milk, with some from CaCO₃. Group 4 was maintained on whey protein isolate/1.2% calcium comprising only protein from whey and calcium mostly from whey with a modest amount from CaCO₃ (e.g., Provon 190). Group 5 was maintained on casein/1.2% calcium, comprising protein from casein and 1.2% calcium from CaCO₃. Group 6 was maintained on soy protein isolate/1.2% calcium, comprising protein from soy and 1.2% calcium from CaCO₃.

Groups 7 and 8 continued on high calcium (1.2%), energy restricted diets. Group 7 used a whey mineral isolate, containing 1.2% calcium and protein from casein without a whey-derived angiotensin converting enzyme (ACE)-inhibitor (TRUE-CAL). Group 8 used a whey mineral isolate with a whey-derived ACE-inhibitor (TRUE-CAL FP).

During the six weeks, body weight, food intake and core temperature were measured weekly. At the end of six weeks, fat pad mass, adipocyte intracellular Ca²⁺([Ca²⁺]ᵢ), fatty acid synthase expression and activity and lipolysis was assessed.

Results

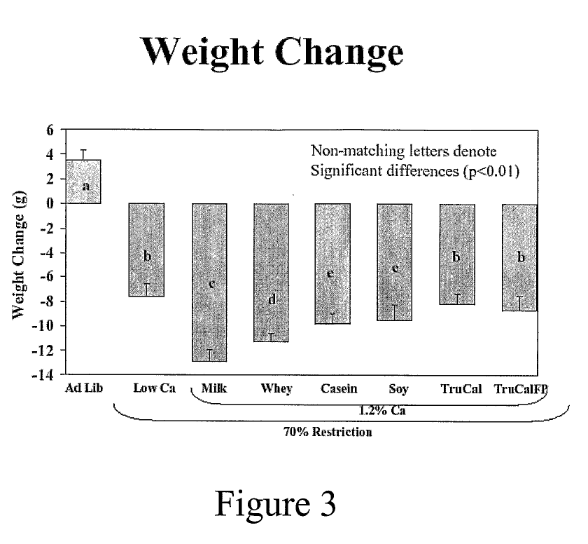

FIG. 3 demonstrates that high calcium milk Group 3 mice had the most significant and substantial weight change, followed by high calcium whey protein isolate Group 4. High calcium casein Group 5 and soy Group 6 had less weight change than milk or whey protein isolate but more than low calcium Group 2 and high calcium whey mineral Group 7 and whey mineral/ACE Group 8. The least amount of weight change was in the Ad Lib Group 1.

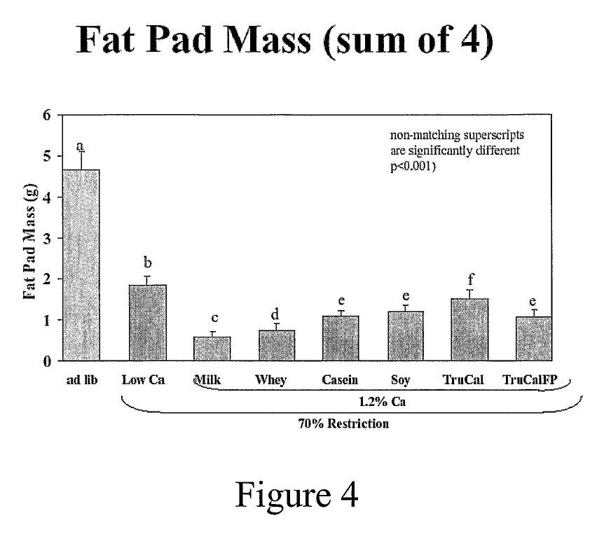

As observed in FIG. 4, Ad Lib Group 1 mice had significantly more fat pad mass than any of the other calcium containing groups. The least fat pad mass was seen in high calcium milk Group 3, followed by high calcium whey protein isolate Group 4. High calcium casein Group 5, soy Group 6 and whey mineral/ACE Group 8 had more fat pad mass than milk or whey protein isolate but less than high calcium whey mineral Group 7, which had even less than low calcium Group 2.

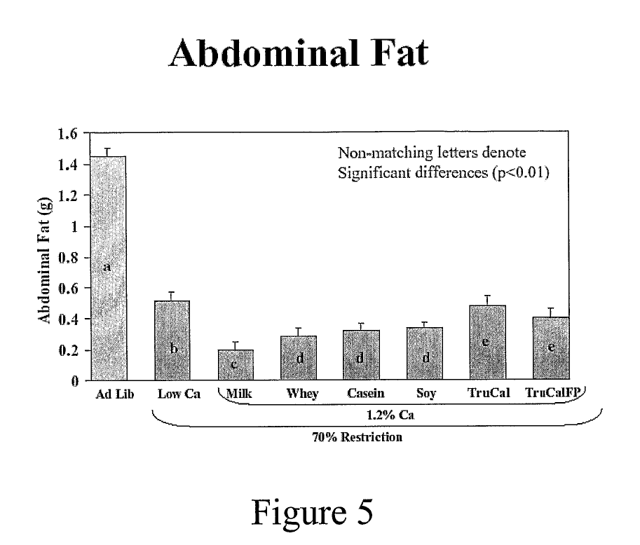

In FIG. 5, Ad Lib Group 1 mice appear to have significantly more abdominal fat than any of the other calcium containing groups. The least abdominal fat was observed in high calcium milk Group 3, followed by high calcium whey protein isolate Group 4, high calcium casein Group 5 and soy Group 6. High calcium whey mineral Group 7 and whey mineral/ACE Group 8 had more abdominal fat than the other high calcium containing groups, but less than low calcium Group 2.

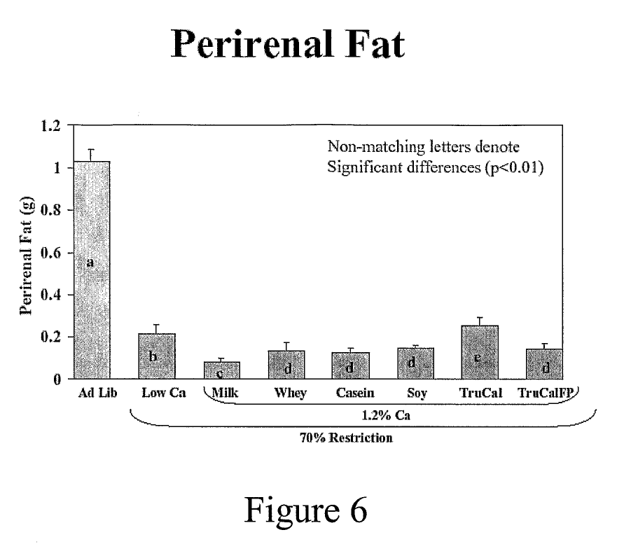

FIG. 6 shows that Ad Lib Group 1 mice appear to have significantly more perirenal fat than any of the other calcium containing groups. The least perirenal fat was observed in high calcium milk Group 3, followed by high calcium whey protein isolate Group 4, high calcium casein Group 5, soy Group 6 and whey mineral/ACE Group 8. Low calcium Group 2 had more perirenal fat than the other high calcium containing groups, but less than high calcium whey mineral Group 7.

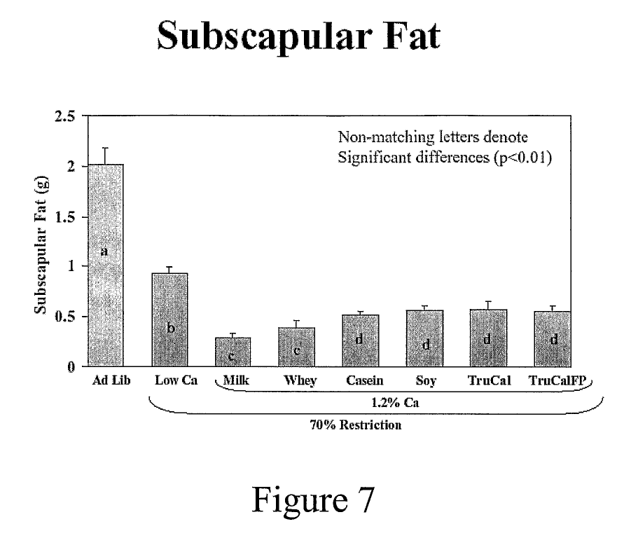

As seen in FIG. 7, Ad Lib Group 1 mice had significantly more subscapular fat than any of the other calcium containing groups. The least subscapular fat was seen in high calcium milk Group 3 and whey protein isolate Group 4, followed by high calcium casein Group 5, soy Group 6, whey mineral Group 7 and whey mineral/ACE Group 8, which had even less than low calcium Group 2.

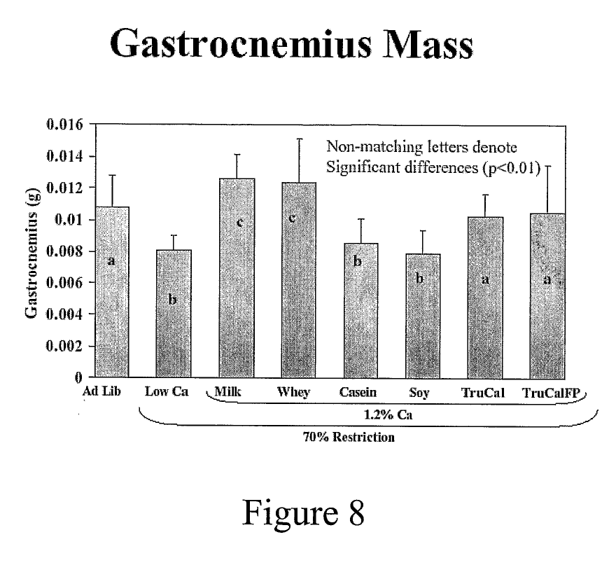

In FIG. 8, high calcium milk Group 3 and whey protein isolate Group 4 had the most gastrocnemius muscle mass, followed by high calcium casein Group 5, soy Group 6 and low calcium Group 2. High calcium whey mineral Group 7 and whey mineral/ACE Group 8 had less than all the other groups, except for Ad Lib Group 1.

Table 2 summarizes these results.

### Conclusion

As demonstrated here, high calcium diets caused a 32% augmentation of fat loss versus the low calcium diet (p<0.0001), while milk augmented this fat loss by 62.5% vs. the other high calcium, diets (p<0.001). Whey was not as effective as milk, and the whey minerals used provided no additional fat loss. Addition of the whey ACE inhibitor reduced fat mass by 22% (p<0.001), although intact milk was still markedly more effective, which may be due to an artifact of processing, resulting in compromised bioavailability. Thus, whey components provide an anti-obesity effect.

In addition, energy restriction on the low calcium diets resulted in a decrease in gastrocnemius muscle mass which was prevented by the whey diet (which produced a modest, significant increase), but not by the calcium-supplemented casein and soy diets.

Whey-based diets appear to promote a repartitioning of dietary energy from adipose tissue to skeletal muscle, resulting in preservation and/or improvement in lean body mass while adipose tissue is lost. Whey products appear to produce the full anti-obesity effect and produced the skeletal muscle sparing effect.

The example shows that inclusion of increased calcium or calcium-rich whey products into the diet of these mice significantly attenuated diet-induced obesity and markedly accelerated both weight and fat loss secondary to caloric restriction. Although these effects were attributable, in part, to calcium, whey-derived sources of calcium exerted greater effect than equivalent amounts of calcium from supplements.

From the foregoing description, one skilled in the art can easily ascertain the essential characteristics of this invention, and without departing from the spirit and scope thereof, can make changes and modifications of the invention to adapt it to various usage and conditions.

Without further elaboration, it is believed that one skilled in the art can, using the preceding description, utilize the present invention to its fullest extent. The preceding exemplary specific embodiments are, therefore, to be construed as merely illustrative, and not limitative of the remainder of the disclosure in any way whatsoever.

The entire disclosure of all applications, patents and publications, cited above and below and in the figures are hereby incorporated by reference.
1. Zemel M B, Shi H, Greer B, DiRienzo D and Zemel P C. Regulation of adiposity by dietary calcium. FASEB J 2000; 14:1132-1138.
2. Shi H, DiRienzo D and Zemel M B. Effects of dietary calcium on adipocyte lipid metabolism and body weight regulation in energy-restricted aP2-agouti transgenic mice. FASEB J 2001; 15:291-293.
3. Pereira M A, Jacobs D R, Van Horn L, Slattery M L, Kartashov A I, Ludwig D S: Dairy consumption, obesity, and the insulin resistance syndrome in young adults. The CARDIA study. JAMA 2002; 287:2081-2089.
4. Zemel M B, Thompson W, Zemel P, Nocton A M, Milstead A, Morris K, Campbell P. Dietary calcium and dairy products accelerate weight and fat loss during energy restriction in obese adults. Am J Clin Nutr 2002; 75 (2S):342S.
5. Zemel M B, Nocton A M, Richards J, Milstead A, Gebhardt, Campbell P J Increasing dairy calcium intake reduces adiposity in obese African-American adults. Circulation 2002; 106 (suppl 2) 11-610.
6. Shi H, Norman A W, Okamura W H, Sen A, Zemel M B: 1,25-dihydroxyvitamin D3 modulates human adipocyte metabolism via nongenomic action. FASEB J 2001; 15:2751-2752.
7. Shi H, Norman A W, Okamura W H, Sen A, Zemel M B. 1a,25-dihydroxyvitamin D3 inhibits uncoupling protein 2 expression in human adipocytes. FASEB J 2002; 16:1808-1810.
8. Teegarden D. Calcium intake and reduction in weight or fat mass. J Nutr 2003; 133:249S-25 1S.
9. Zemel M B. Mechanisms of dairy modulation of adiposity. J Nutr 2003; 133:252S-256S.
10. Heaney R P. Nonnalizing calcium intake: Projected population effects for body weight. J Nutr 2003; 133:268S-270S.
11. Melanson E L, Shart T A, Schneider J, Donahoo W T, Grunwald G K, Hill J O. Relation between calcium intake and fat oxidation in adult humans. Int J Obesity 2003; 27:196-203.

1. A method comprising administering to an individual regulating body weight a calcium containing whey product in an amount effective to maintain muscle mass while inducing weight and/or fat loss, preventing weight and/or fat gain, and/or increasing the metabolic consumption of adipose tissue in the individual.

2. The method of claim 1, wherein the individual is maintained on a caloric restricted diet.

3. The method of claim 1, wherein the muscle is skeletal muscle.

4. The method of claim 1, wherein the muscle is limb muscle.

5. The method of claim 1, wherein the administered whey product selectively induces loss of fat pad mass, abdominal fat, perirenal fat or subscapular fat and combinations.

6. The method of claim 1, wherein the whey product induces increase in muscle mass.

7. The method of claim 1, wherein the whey product comprises whey-derived protein isolate, whey-derived mineral fraction, whey-derived enzyme-inhibiting peptide fraction, and/or combinations.

8. The method of claim 7, wherein the whey-derived protein isolate comprises bovine serum albumin, alpha-lactoglobulin, beta-lactoglobulin, kappa-casein fragment 106-109, lactoferrin, lactoperoxidase, immunoglobulins.

9. The method of claim 7, wherein the whey-derived mineral fraction comprises calcium, copper, magnesium, phosphorus, potassium, selenium, zinc and/or combinations.

10. The method of claim 7, wherein the whey-derived mineral fraction comprises calcium.

11. The method of claim 1, wherein the whey product fortifies a dairy product.

12. The method of claim 11, wherein the dairy product is milk, yogurt or cheese.

13. The method of claim 1, wherein the whey product comprises an enzyme-inhibiting peptide fraction.

14. The method of claim 13, wherein the enzyme-inhibiting peptide comprises angiotensin converting enzyme (ACE) inhibiting peptide.

15. The method of claim 1, wherein the whey product is in the form of a powder.

16. The method of claim 1, wherein the whey product is incorporated into a nutritional or dietary composition or supplement.

17. The method of claim 1, wherein the whey product is incorporated into a food product.

18. The method of claim 17, wherein the food product is a beverage.

19. The method of claim 17, wherein the food product is selected from the group consisting of acidic juice beverages, acidic beverages, neutral pH beverages, nutritional supplement foodstuffs, confectionery products, dairy products, bakery products and farinaceous products.

20. The method of claim 1, wherein the whey product is in the form of a tablet, capsule, or combination with other minerals and/or vitamins.

21. The method of claim 1, further comprising administering the whey product to an individual and thereby treating a disorder selected from the group consisting of high blood pressure, stroke, obesity, kidney stones, colon cancer, breast cancer, head and neck tumors, premenstrual syndrome, postpartum depression, hypertensive disorders of pregnancy, Type-2 diabetes, depression, asthma, inflammatory bowel disease, attention deficit disorder, migraine headaches, kidney disease, hypercholesterolemia, congestive heart failure, and immune deficiency.

22. The method of claim 1, wherein the whey product is administered to an individual in need thereof to treat obesity.

23. A method of treating a body weight condition comprising administering to an individual a therapeutically effective amount of a nutritional composition having two or more weight control components selected from the group consisting of whey-derived mineral fraction, whey-derived protein isolate, and whey-derived enzyme-inhibiting peptide fraction, to maintain muscle mass while inducing weight and/or fat loss, preventing weight and/or fat gain, and/or increasing the metabolic consumption of adipose tissue in the individual.

24. A weight management composition comprising therapeutically effective amounts of two or more weight control components selected from the group consisting of whey-derived mineral fraction, whey-derived protein isolate and whey-derived enzyme-inhibiting peptide fraction, in a package labeled with information about the effects of consuming effective amounts of the components to maintain muscle mass while inducing weight and/or fat loss, preventing weight and/or fat gain, and/or increasing the metabolic consumption of adipose tissue in the individual.
